(19) United States
(12) Patent Application Publication
(10) Pub. No.: US 2021/0259255 A1
(43) Pub. Date: Aug. 26, 2021

(54) Immobilization of Plant Growth Promoting Microorganisms in Hydrophobic Carriers

(71) Applicant: Battelle Memorial Institute, Columbus, OH (US)

(72) Inventors: Katarzyna H. Kucharzyk, Columbus, OH (US); Jeff Cafmeyer, Columbus, OH (US); Veronica Fulwider, Columbus, OH (US); Anthony D. Duong, Columbus, OH (US)

(21) Appl. No.: 17/183,157

(22) Filed: Feb. 23, 2021

Related U.S. Application Data
(60) Provisional application No. 63/052,456, filed on Jul. 15, 2020.
(60) Provisional application No. 62/980,413, filed on Feb. 23, 2020.

(51) Int. Cl. A01N 63/27; C12N 1/20; A01N 25/26; A01N 25/10

(52) U.S. Cl. CPC A01N 63/27; C12N 1/20; C12R 2001/38; A01N 25/10; A01N 25/26

(57) ABSTRACT

The invention provides an encapsulated microorganism comprising particles comprising a core and a coating over the core. The core comprises a microorganism and an alginate or polyaspartate. The coating comprises a lipid-based coating. The application of lipid waxes serves to repel diffusion of any water-soluble chemicals that may be present on the surface of commercially available seeds. The lipid waxes applied on top of encapsulated microbial capsules are degradable by enzymes produces by germinating seeds. Thus, the colonization of rhizosphere of germinating plant by the commensal bacteria is a time targeted process to the benefit of the plant.

### RELATED APPLICATIONS

This application claims the priority benefit of U.S. Provisional Patent Applications Ser. Nos. 63/052,456 filed 15 Jul. 2020 and 62/980,413 filed 23 Feb. 2020.

### INTRODUCTION

Immobilization and encapsulation of bacterial cells has been widely used in agriculture, pharmaceutical, food, and other industries to achieve a protective structure or a capsule allowing immobilization, protection, release, and functionalization of active ingredients. Therefore, encapsulation allows for less exposure to adverse environmental factors, tends to stabilize cells, potentially enhancing their viability and stability in the production, storage, and handling of cultures and also confers additional protection during rehydration. However, despite the long history of inoculation of seeds with plant growth promoting microbial species and clear laboratory demonstration of the ability of a wide range of other beneficial microorganisms to improve crop performance, there are still very few commercially available microbial inoculations that stay viable on seed. Further research is needed before the benefits of a wide range of environmentally sensitive potential seed inoculants can be captured for use in agriculture, ecosystem restoration and bioremediation. There is no single solution to the challenge of improving the ability of seed inoculants to establish and function consistently in the field.

Thus, there remains a need for development of novel formulations that maintain the viability of both inoculant and seed during storage.

Plant growth promoting bacteria can be used to ameliorate environmental challenges. For example, Pseudomonas putida can significantly enhance growth of wheat under heat stress. Some Bacillus subtilis produce cytokinin, a plant hormone that interferes with drought-induced suppression of shoot growth thereby enhancing plant growth throughout periods of drought. Under stressful conditions plants can produce the substance ACC, a precursor to the hormone ethylene which stunts plant growth. Bacterium serratia produces an enzyme that breaks down ACC resulting in better plant growth. Many Pseudamonas and Bacillus isolates have insecticidal activity. Various bacteria including Pseudamonas fluorescens produce antibiotic compounds like pyrrolnitrin which confers resistance to various fungal pathogens such as Rhizoctonia solani which causes damping-off disease in cotton. Siderophore producing bacteria, such as Microbacterium and Pseudomonas, can bind heavy metals and reduce toxicity to plants. Commercial examples include: Cell-Tech® (rhizobia), TagTeam® (rhizobia and Penicillium bilaiae), Nodulator® (Bradyrhizobium japonicum), Nodulator® PRO (Bradyrhizobium japonicum and Bacillus subtilis), Accomplish® (PGPR+enzymes+organic acids+chelators), Bioboots® (Delftia acidovorans), and^{Bioboots}® (soybean)(Delftia acidovorans and Bradyrhizobium sp.).

### SUMMARY OF THE INVENTION

The invention provides an encapsulated microorganism, comprising particles comprising a core and a coating; wherein the core comprises: a microorganism and/or an enzyme; and an alginate or polyaspartate; and the coating comprises a lipid-based coating. In various embodiments, the invention can be further characterized by one or any combination of the following: wherein the core further comprises a polyelectrolyte; wherein the core comprises a Pseudomonas bacteria; wherein the core further comprises a lipid; wherein the lipid-based coating comprises a naturally occurring wax; wherein the naturally occurring wax is selected from the group consisting of carnuba wax, sorghum wax, candelilla wax, rice bran wax, soy wax, and combinations thereof; wherein the lipid-based coating comprises hydrogenated soybean oil blended with stearic acid; wherein the lipid-based coating contains at least 95 wt % lipids; wherein the alginate or polyaspartate comprise calcium alginate, sodium alginate, manganese alginate, zirconium alginate, calcium poly(aspartate), manganese poly(aspartate), zirconium poly (aspartate), and combinations thereof (with alginates being particularly preferred); wherein the alginate or polyaspartate is an alginate cross-linked by MnCl2, BaCl2, CaCl2, or combinations thereof; wherein the encapsulated microorganism and/or enzyme is in the form of particles and wherein at least 90% of the particles are between 1 and 100 μm in diameter; wherein the encapsulated microorganism and/or enzyme is in the form of particles and wherein at least 70% of the particles are between 1 and 10 μm in diameter; wherein the core comprises nitrogen-fixing bacteria, endo and ectomycorrhizal fungi, and/or plant growth promoting bacteria; wherein the core comprises: Rhizobium tropici, Rhizobium leguminosarum, Sinorhizobium meliloti, Pseudomonas protegens, Burkholderia phytofirmans, Bradyrhizobium japonicum, Azospirillum brasilense, Rhodopseudomonas acidophila Rhodotorula sp., and/or Penicillium bilaiae; wherein the encapsulated microorganism and/or enzyme is in the form of particles and wherein, as compared to the uncoated particles, the microoganism's activity is 10 times higher or 100 times higher than the uncoated particles after 8 weeks or after 15 months of storage at standard conditions (20° C. in air, 1 atm, no humidity); wherein the core comprises skim milk, trehalose or maltodextrin; wherein the encapsulated microorganism of any of the preceding claims in the form of a coating disposed on a seed.

In another aspect, the invention comprises a seed coated with the encapsulated microorganism. Preferred seed types are corn, wheat, soy bean, or cotton. The invention also includes a method of growing a plant comprising putting one of the seeds of claims into soil.

In a futher aspect, the invention comprises a method of making an encapsulated microorganism, comprising: dropping a solution of the microorganism (or combination of microorganisms) and an alginate and/or polyaspartate into a stirred bath containing a cross-linking solution to form particles comprising the microorganism and a cross-linked alginate or polyaspartate; and coating the resulting particles with a lipid-based wax so that the lipid waxes form an outer shell over the alginate or polyaspartate of the core particle.

As is standard terminology, comprising means including and, in any of its aspects, the invention may by alternatively be described by the more limiting phrases consisting essentially of or consisting of.

A problem addressed by this invention is the need to protect microorganisms encapsulated in calcium alginate or related materials from diffusion of any chemical compounds that may inhibit or stop their growth. The application of lipid waxes serves to repel diffusion of any water-soluble chemicals that may be present on the surface of commercially available seeds. The lipid waxes applied on top of encapsulated microbial capsules are degradable by enzymes produced by germinating seeds. Thus, the colonization of rhizosphere of germinating plant by the commensal bacteria is a time targeted process to the benefit of the plant.

The invention can provide advantages such as: (1) allowing lipid waxes to repel aqueous chemicals of detrimental effect to microorganisms; (2) allowing lipid waxes to prevent desiccation of water content entrapped in the calcium alginate—microorganisms capsules.

### BRIEF DESCRIPTION OF THE FIGURES

FIG. 1 illustrates a process in which microorganisms and alginate are sprayed into a collection bath where the alginates are cross-linked and formed encapsulated microorganisms. The capsules may contain additional components such as adjuvants and biodegradable polymer inside a hydrophobic shell. At far right is a photomicrograph showing the spherical bodies.

FIG. 2 shows a photomicrograph of a particle of the core encapsulated in a wax coating. The particle has a diameter of about 25 μm.

FIG. 3 is an SEM image of a particle of the core encapsulated in a wax coating.

FIG. 4 shows viability results of free and encapsulated (encap) samples of microorganisms identified for testing during an 18-month study. For each microorganism, viability is shown in colony forming units (cfu) as a function of time from left to right. 12 months of testing is plotted on the graph.

FIG. 5 shows viability results of free and encapsulated (encap) samples of microorganisms identified for testing during an 18-month study. First 10 months of testing is plotted on the graph.

FIG. 6 Infrared spectra showing soy wax degradation by seed produced esterase enzymes. The appearance of a peak at 1035 cm⁻¹ in the T1 (middle) and T2 (top) samples. A peak in this region is generally associated with C—O bonding. The C═O bond associated with the ester is still prominent (1734 cm⁻¹) and appears to not have changed in intensity from sample to sample.

FIG. 7 shows the viability of free and alginate encapsulated microorganisms with skim milk.

FIG. 7 shows the viability of free and encapsulated P. fluorescens with exopolysaccharide and skim milk.

FIG. 8 shows the viability of P. protegens encapsulated in the inventive system including a lipid outer coating and incubated in 5 different chemical seed coating materials. The inventive encapsulated and on seed applied microorganisms show survival even in presence of biocidal chemicals.

FIG. 9 shows sprouting of a seed that has been treated with the encapsulated microorganisms. At right is a photo of a greenhouse trial with the coated seeds (left) and controls (right).

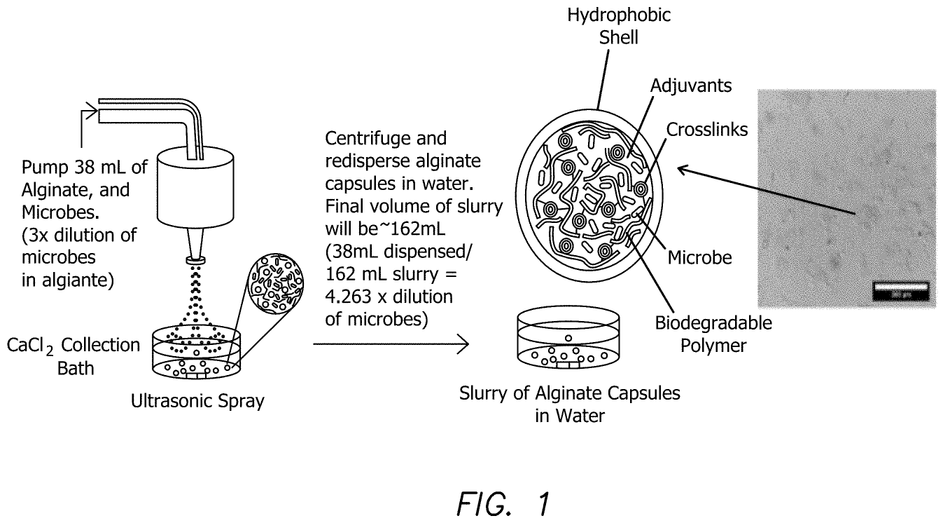

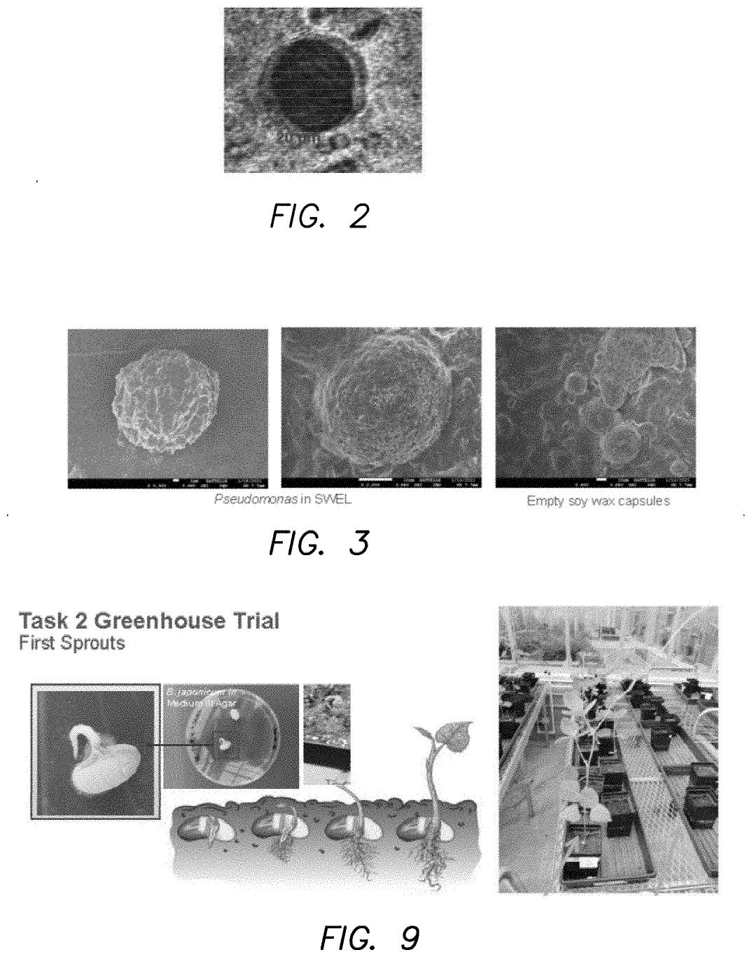

### DETAILED DESCRIPTION OF THE INVENTION

The core comprises an encapsulated microorganism(s) and/or enzyme. In addition to the microorganism and/or enzyme, the core may comprise an alginate or polyaspartate or polyelectrolyte. The core may also contain some lipid as is present in the shell. Preferred members of the alginate or polyaspartate comprise calcium alginate, sodium alginate, manganese alginate, zirconium alginate, calcium poly(aspartate), manganese poly(aspartate) and zirconium poly (aspartate); with alginates being particularly preferred. These may be cross-linked, for example by the addition of MnCl₂, BaCl₂, and/or CaCl₂).

The size of the microorganism-containing core is preferably in the range of 1 and 10 μm or 2-6 μm or 3-5 μm in diameter (at least 90% or at least 80% or at least 70% or at least 50% of the particles (by number); this can be measured by microscopy) or, alternatively, in the range of 25-100 μm, 25-50 μm, or 50-100 μm in diameter (at least 90% or at least 80% or at least 70% or at least 50% of the particles (by number); this can be measured by microscopy).

The microorganisms preferably impart properties to the rhizosphere such as: biocontrol agent; promoting plant growth (for example, a fungus that promotes growth), enhance phosphorus (P) availability, promote P mobilization from organic sources, promote mobilization of selenium from organic sources, and/or nitrogen-fixation. Preferred organisms include nitrogen-fixing bacteria, endo and ectomycorrhizal fungi, and/or plant growth promoting bacteria. Examples include Pseudomonas fluorescens, Penicillium bilaiae, and Bradyrhizobium japonicum. (see table 3 for specific microbial genera tested)

The lipid-based coating preferably comprises naturally-occurring wax or waxes, examples include carnuba wax (long chain wax esters, high hardness, high melting point), sorghum wax (long chain alcohols and long chain aldehydes (low ester content), high hardness, high melting point), candelilla wax (was esters, higher amount of free fatty acids), rice bran wax (long chain wax esters, high hardness, high melting point), and/or soy wax; preferably made up of an ester of a long-chain alcohol and a fatty acid, typically, the fatty acid comprises 4 to 24 carbons and the long-chain alcohol comprises 12 to 32 carbons. Soy wax is preferably hydrogenated soybean oil (triglyceride fatty acids) blended with stearic acid and optionally oil to have a melting point similar to paraffin waxes (40 to 70° C.).

The lipid waxes are preferably highly hydrophobic and repel water disabling diffusion of liquids through the capsule and protecting the capsule core containing microorganisms. Capsules containing microorganisms are preferably between 10-100 μm diameter—average size of the microbial cell is 20-50 μm diameter. The waxes typically have the advantage of being degradable by the enzymes produced by the germinating seed; this was observed by the proteomic analysis of germinating seed. The coating on the exterior of the particles can be at least 95 wt % or at least 99 wt % lipids. The coating is hydrophobic and repels water, disabling diffusion of liquids through the capsule and protecting the core that contains microorganisms. The lipid waxes prevent desiccation of water content entrapped in the microorganism-containing core. The lipid-based coating can also repel components of commercial seed coat formulations that are toxic to the microorganisms. The lipid waxes are degradable by the enzymes e.g., esterases, produced by the germinating seed. This phenomenon was proven by proteomic analysis of germinating seed.

The coating can prevent desiccation of the microorganism. The coating was surprisingly found to maintain activity of the microorganisms as compared to the uncoated particles—preferably activity is 10 times higher or 100 times higher than the uncoated particles after 8 weeks or after 15 months of storage at standard conditions (20° C. in air, 1 atm, no humidity).

Preferably, the particles are spherical (see figures). The coated particles are preferably between 1 and 100 μm, or 10 to 100 μm, or more preferably between 20 and 50 μm in diameter (at least 90% or at least 80% or at least 70% of the particles (by number); this can be measured by microscopy). Capsules containing microorganisms are preferably between 10-100 um diameter—average size of the microbial cell is 3-5 um diameter.

If desired, the particles may contain ordered layers to control the timed release of desired microorganisms in conjunction with their need in the rhizosphere. The particles can be in emulsions or aqueous suspensions.

The invention also includes a seed composition comprising seeds coated with, and/or mixed with, the encapsulated microorganism powder. Seeds can be any plant seeds; for example, corn, wheat, soy beans, and cotton.

The invention also includes a method of growing a plant comprising putting one of the seeds coated with the encapsulated microorganism into the soil. The plant can be raised conventionally, for example by watering the seed in the soil. The lipid-based coating is degradable by enzymes produced by germinating seeds (this was proven by proteomic analysis of the germinating seed). Thus, the colonization of the rhizosphere of the germinating plant by the commensal bacteria is a time-targeted process to the benefit of the plant. Use of the inventive coatings can produce higher yields and/or faster growth; and may reduce the need for fertilizer and/or pesticides.

The invention also includes methods of making the encapsulated microorganism powder in which a solution of the microorganism (or combination of microorganisms) and the alginate and/or polyaspartate is dropped into a stirred bath containing a cross-linking solution. Cross-linking agents may comprise, for example, MnCl2, BaCl2, and/or CaCl2. Cross-linking of the bead can be varied to control microbe release and shelf stability. Additional synthetic details may be obtained from US Patent Pub. No. 2018/0142229 by Kucharzyk et al. which is incorporated herein as if reproduced in full below.

The resulting particles are then coated with the lipid-based wax. Conventional methods can be used for the coating step, for example, dripping or spraying onto the surface of the alginate or polyaspartate. The lipid waxes form an outer shell over the alginate or polyaspartate of the core particle. Incorporation of the lipid waxes into the microbial core is also possible.

The invention, in some embodiments, can be characterized by possessing one or any combination of the properties described; or within ±50%, 30%, or within ±10% of one or any combination of the properties, compositions, or other features described herein. The properties include any of the measurements provided in the data reported herein. For example, the invention includes an encapsulated microorganism having enhanced viability such that when tested by microbial plate counting methods, the microorganism exhibits at least 10E6 colony forming units after 1 week or 4 weeks or 12 months, or a 100× or less or 10× or less loss in viability after 1 week or 4 weeks or 12 months when stored at about 20° C. and 50% relative humidity (this property can be measured as the powder or a coating on a seed). Alternatively, the inventive encapsulated microorganism can be characterized as having a 2× greater, 5× greater, or 10× greater viability than the same composition without the lipid coating after storage for 4 weeks or 12 months at about 20° C. and 50% relative humidity (measured as the powder or a coating on a seed).

### Examples

A literature review of references discussing organisms of recent interest to agriculture was performed and organisms listed in the Table 1 were down selected for encapsulation and stability testing. Table 2 lists ATCC and DSMZ available strains purchased for this program.

TABLE 1
Microorganisms down selected for testing

| Microorganism | Target seed | Function | Reference |
|---|---|---|---|
| Rhizobium tropici | Rice | Si solubilizing and release for plant | Chandrakala |
| Rhizobium sp. CNNWYC119 | | growth promotion (PRP) | 2019 |
| Sinorhizobium meliloti | Wheat | Enhancement of root expression via | Moshynets 2019 |
| Pseudomonas aeruginosa | | production of acyl-homoserine | |
| | | lactones (AHLs); quorum sensing | |
| Pseudomonas protegens | Fruit crop | Antifungal | Andreolli 2019 |
| DSM 19095 | | | |
| Pseudomonas protegens MP12 | | | |
| Burkholderia phytofirmonas PSJN | | | |
| Co-inoculation with | Soy | Symbionts of legumes, participating in | |
| Bradyrhizobium japonicum | | nitrogen fixation and having an | |
| Azospirillum brasilense Ab-V5 | | important impact on the protein | |
| | | content of soybeans | |
| Rhodopseudomonas acidophila | Stress | EPS producing and salt retaining, P | Silambarasan |
| Rhodotorula sp. CAH2 | conditions of | binding and retaining | 2019 |
| | many | | |
| | commodity | | |
| | crops | | |

TABLE 2
Microorganisms down selected for purchase and testing

| Species Name | Vendor | Catalog # |
|---|---|---|
| Rhizobium tropici | DSMZ | 11418 |
| Rhizobium leguminosarum Jordan | ATCC | 10004 |
| Sinorhizobium meliloti | ATCC | 9930 |
| Pseudomonas protegens DSM 19095 | DSMZ | 19095 |
| Burkholderia phytofirmans PSJN | DSMZ | 17436 |
| Bradyrhizobium japonicum | ATCC | 55749 |
| Azospirillum brasilense Ab-V5 | ATCC | 29729 |
| Rhodopseudomonas acidophila | DSMZ | 137 |
| Rhodotorula sp. | DSMZ | 28479 |
| Penicillium bilaiae | ATCC | 18309 |

Each down selected strain listed in Table 2 was first inoculated agar following conditions specifically defined by the ATCC or DSMZ culture collection. Well-developed cultures were then inoculated in a specifically defined liquid medium prepared according to the ACTT or DSMZ culture collection instructions.

Solid and liquid media are for each strain are defined in Table 3 below and instructions can be downloaded from the ATCC or DSMZ websites.

TABLE 3
Solid and liquid media required for propagation of down selected microorganisms.

| | | Catalog | Liquid Growth | Solid Growth |
|---|---|---|---|---|
| Species Name | Vendor | # | Media | Media |
| Rhizobium tropici | DSMZ | 11418 | Peptone-Yeast | Yeast Extract- |
| | | | Extract Medium | Mannitol Agar |
| Rhizobium leguminosarum | ATCC | 10004 | Peptone-Yeast | Yeast Extract- |
| Jordan | | | Extract Medium | Mannitol Agar |
| Sinorhizobium meliloti | ATCC | 9930 | Peptone-Yeast | Yeast Extract- |
| | | | Extract Medium | Mannitol Agar |
| Pseudomonas protegens | DSMZ | 19095 | Nutrient Broth | Nutrient Agar |
| Burkholderia phytofirmans | DSMZ | 17436 | Trypticase Soy Broth | Trypticase Soy Agar |
| Bradyrhizobium japonicum | ATCC | 55749 | Peptone-Yeast | Yeast Extract- |
| | | | Extract Medium | Mannitol Agar |
| Azospirillum brasilense | ATCC | 29729 | Spirillum Nitrogen | Spirillum Nitrogen |
| | | | Fixing Medium | Fixing Agar |
| Rhodopseudomonas | DSMZ | 137 | Rhodospirillaceae | Rhodospirillaceae |
| acidophila | | | Medium | Agar |
| Rhodotorula sp. | DSMZ | 28479 | Potato Dextrose Broth | Potato Dextrose Agar |
| Penicillium bilaiae | ATCC | 18309 | Malt Extract Broth | Malt Extract Agar |

### On Seed Application of Encapsulated Microorganisms

After encapsulation, each culture or combination of cultures was applied onto the soybean seeds. Maltodextrin was used as a sticker and were added to the encapsulated formulation of microorganisms prior to seed coating. Three 100-gram batches of seed were coated for each organism and then mixed to form one homogenous batch that was equally split into two paper-backed autoclave pouches. One pouch of each was stored at room temperature, one at medium temperature and medium humidity and one at higher temperature and higher humidity.

At T=0 when each batch is made a sample of the seed and of the liquid formulation will be extracted in 3 mM sodium citrate and plated for enumeration on medium that supports growth of the organism.

### Long Term Storage of Liquid Cultures, Encapsulated Cultures, and Coated Seeds

Storage conditions per each group of microorganisms were as follows:
Liquid microbial cultures—18 months stability testing, 3 replicates per time point pulled each month, room temperature storage, viability testing by plating and calculating CFUs of culture aliquots
Encapsulated microbial cultures—18 months stability testing, 3 replicates per time point pulled each month, room temperature storage, viability testing by dissolving calcium alginate shell, plating and calculating CFUs of culture aliquots.
On seed applied encapsulated microbial cultures—9 months stability testing, 3 replicates per time point pulled each month, viability testing by dissolving calcium alginate shell, plating and calculating CFUs of culture aliquots
   Storage in:
      50 F 50% RH
      75 F 65% RH

Viability of microorganisms was evaluated at each time point during the 18 months of long-term stability testing. Some microbial formulations were pulled and saved for molecular biological diagnostics at points rendered of value to the project.

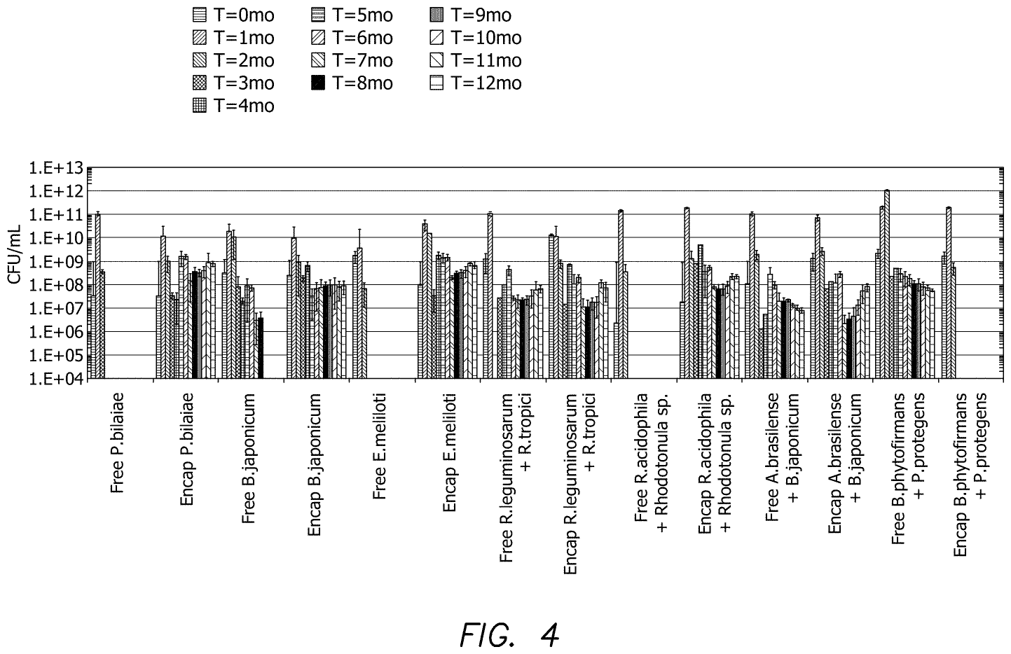

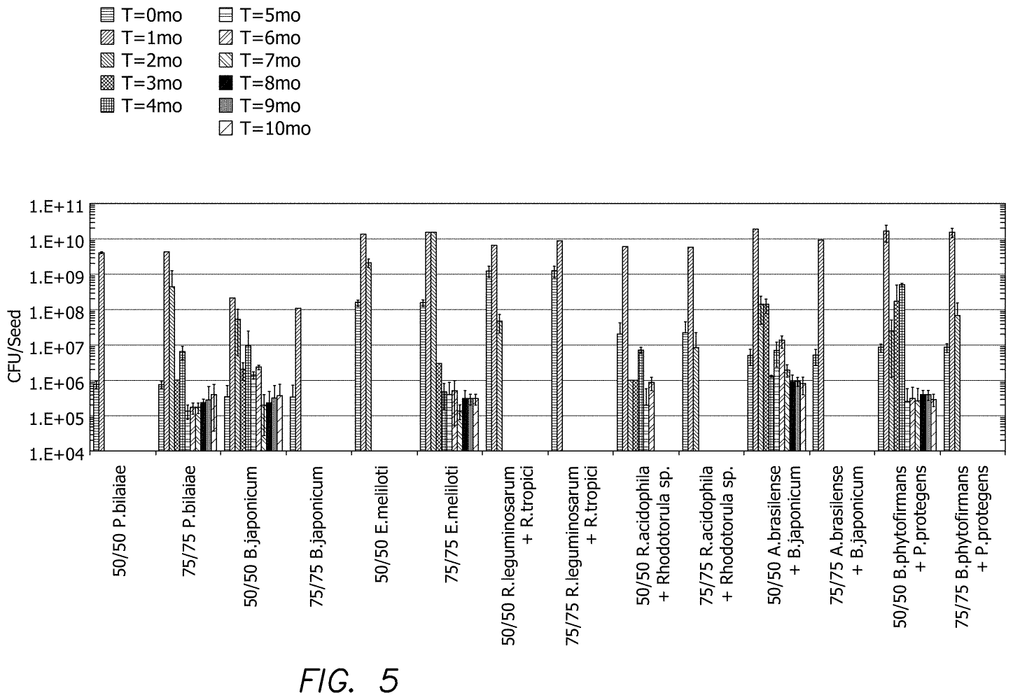

The viability testing for microorganisms specified in Table 3 has provided insights into stability of the microbes in free, unencapsulated form and encapsulated on shelf. The data is provided in FIG. 4. The viability testing of microorganisms specified in Table 3, encapsulated, and applied onto the clean, with no chemical seed coating materials present, soybean seeds is shown in FIG. 5.

Application of Lipids for Protection from Chemical Seed Coating Materials.

The next steps in the experimentation is application of lipids to prevent diffusion of chemical seed coating materials in biologicals. We utilized naturally occurring lipids, comprising of one or more lipid types, to stabilize microorganisms (bacteria and fungi) and proteins (peptides) for on seed application and plant growth promotion. The lipid-based stabilizers tested are the lipids and/or waxes or combinations of:
Carnauba wax—long chain wax esters, high hardness, high melting point,
Sorghum wax—long chain alcohols and long chain aldehydes (low ester content), high hardness, high melting point,
Candelilla wax—wax esters, higher amount of free fatty acids,
Rice bran wax—long chain wax esters, high hardness, high melting point,
Soy wax—hydrogenated soybean oil (triglyceride fatty acids), blended with oil and stearic acid to have melting point like paraffin waxes.

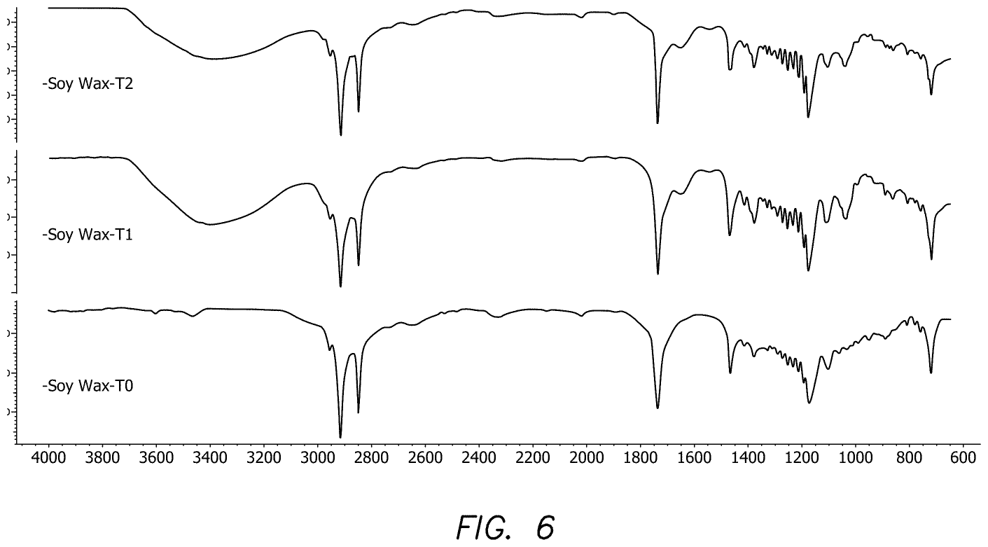

The data showed that the seed released enzymes such as esterases degrade soy waxes with a visible change in their spectra (FIG. 6) and viscosity. Thus, soy waxes are carried through the next steps of the program to provide protection to the encapsulated microorganisms from biocidal effects of chemical seed coating agents present on variety of pre-treated seeds. Three scenarios to test methods of wax application into the encapsulated formulations were tested:
1) Application of wax emulsion into the alginate solution during dripping procedure. This scenario produces capsules with soy wax embedded through the structure of the capsule.
2) Application of wax emulsion in a twostep process and after the calcium alginate capsules have been already produced. This scenario allows for an even coating of the external surface of the capsules and provides protection from desiccation of the microbials but also back diffusion of CSC inside the capsule.
3) Application of wax emulsion as in scenario 2 with dilution and coapplication of maltodextrin in the wax emulsion onto the surface of the seed.

### Skim Milk

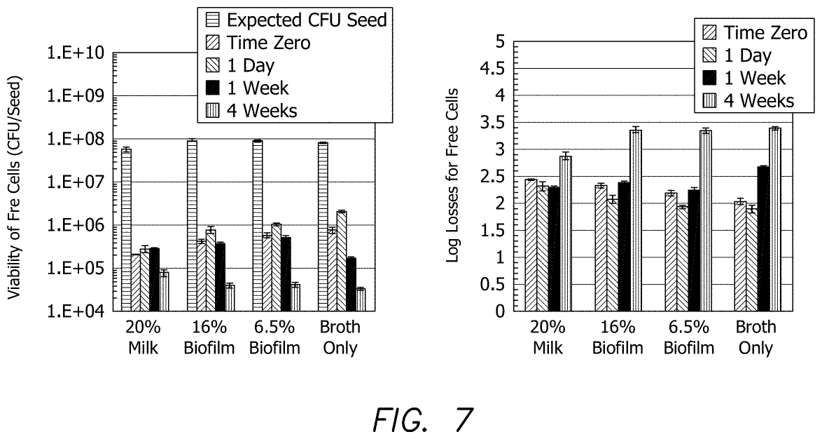

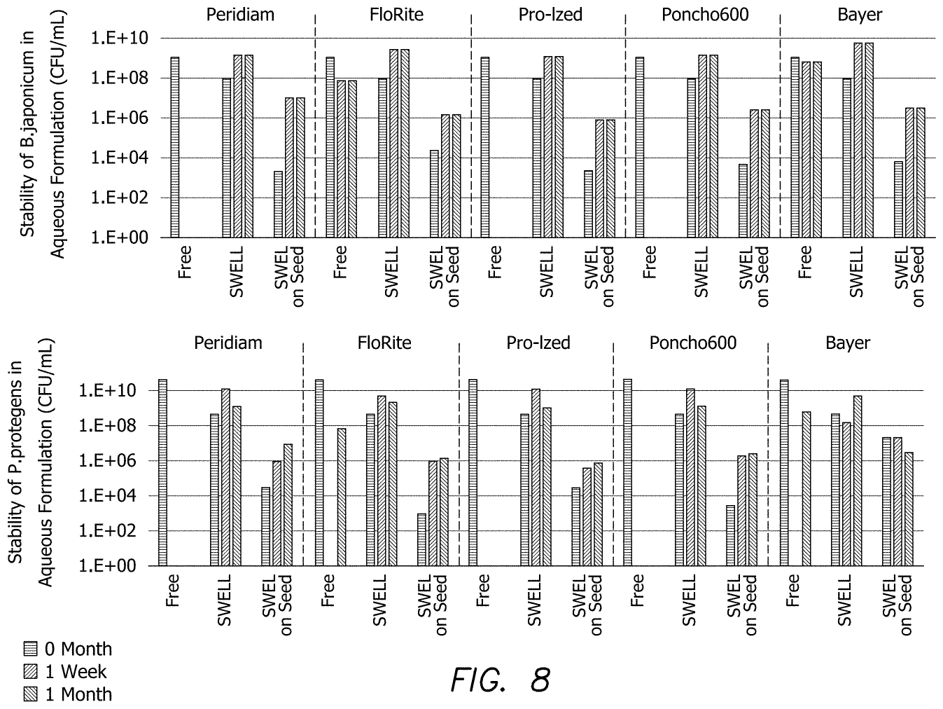

Skim milk is an additive used in bioformulations to enhance cell viability after storage found that the addition of skim milk powder to alginate-encapsulated Azospirillum brasilense significantly increased the cell number within the cell-bead structure. FIG. 7 shows stability testing. After 12 months of storage, 100% recovery of viable cells was obtained from skim milk-alginate encapsulated microorganisms.

Testing P. fluorescens in Amendments with Skim Milk

Pseudomonas fluorescens was prepared by concentrating via centrifugation targeting 2×10e11 cfu/mL. and then re-suspending in broth, broth plus 16% biofilm, broth plus 6.5% biofilm and broth plus 20% milk. To do this 2 mL of bacteria was aliquoted into four microfuge tubes and centrifuged down (5 minutes) in a microcentrifuge (10,000 RPM, 9300 relative centrifugal force, rcf). The supernatant was removed, and the volume was measured to calculate the remaining volume of the pelleted bacteria. The bacteria did not pellet well, and volumes ranged from 680-740 μL. To each pellet a volume of supernatant broth, broth plus 50% biofilm and broth plus 25% biofilm was added back targeting a final volume of 1 mL. For the 20% milk sample, broth was added to bring the volume to 1 mL and then 0.2 grams of milk powder was added to give a final concentration of 20%. The CSC and microorganisms were applied to 100 grams of seed using the two-step jar process. A timer was used to regulate each coating step. CSC was first applied to the top side wall of a jar while rotating and then 100 grams of corn seed was added. The jar was capped and rotated/shaken for ˜10 seconds. The jar lid was opened and 200 μL of microbe formulation was applied to the upper side wall while rotating the jar and then recapped and rotate/shaken for an additional ˜30-60 seconds to distribute the microbe on the seed. Seeds treated with only microbe (no CSC) were treated similarly, except rotated/shake for 40-70 seconds.

Volumes of specific CSC and microbial treatment applied are listed below:
a. Sample 42: 850 μL CSC+200 μL free microbe in 20% milk at 2×10e11 cfu/ml
b. Sample 43: 850 μL CSC+200 μL free microbe in 16% biofilm at 2×10e11 cfu/ml
c. Sample 44: 850 μL CSC+200 μL free microbe in 6.5% biofilm at 2×10e11 cfu/ml
d. Sample 45: 850 μL CSC+200 μL free microbe in broth at 2×10e11 cfu/ml

After application of CSC and microorganism the seed samples were poured onto aluminum foil and allow to air dry in a biosafety cabinet until dry (˜60-90 minutes). After drying the seeds were place into autoclave pouches (plastic on one side paper on the other) and stored at room temperature. Seed samples were extracted after the drying step and enumerated by following the steps below:
a. Place 25 corn seeds with CSC-microbial coating into a 50 mL tube.
b. Add 25 mL sterile water.
c. Vortex samples for 20 minutes to extract the microbes from the seed.
d. Dilute samples serially 1:10 in PBS and plate in triplicate for enumeration (10⁻⁴-10⁻⁸ final on plate).

On day zero the 2×10e11 CFU/mL formulations were plated to confirm the concentration of the inoculum. The seed extraction and plating was repeated at 1 day, ˜1 week and 4-week time points. The seeds were extracted in water on day zero as has typically been done in the past. The results are shown in FIG. 7.

### Application of Soy Waxes to the Encapsulated Formulations

### Capsule Morphology Study

Pseudomonas fluorescens culture was grown to optical density OD=1 and encapsulated in calcium alginate and SWEL. Morphology testing of the encapsulated microorganisms was performed to determine the structure function relationship of the current SWEL technology. Previous efforts have identified a formulation and processing method to yield encapsulated microbes with improved stability against chemical seed treatments both on seed and in liquid formulation. This is a big technical improvement as these chemical seed treatments contain antimicrobials that will kill most (if not all) of the plant growth promoting rhizobium of interest.

We used fluorescent dyes chosen to partition selectively into the hydrophilic alginate core, and other dyes chosen to partition into the lipophilic wax coating. We may also select microbes that have been genetically modified to fluoresce. The images provide understanding of where the microbes are, and the degree of coating attained by the lipid layer.

Microbial Survival with Selected Pesticides—Short Term Testing

Pseudomonas fluorescens culture was tested in time course experiment with variety of commercially available chemical seed coating mixtures to assess viability of microorganisms in SWEL. Microorganisms were encapsulated in SWEL and applied onto the corn seeds or soybean seeds. Time points for free, encapsulated SWEL on shelf and on seed microorganisms will be taken at selected intervals and viability of the culture will be assessed to define the protective nature of wax encansulatinn (SWEL) against other chemical seed coating substances (CSCs).

| Microorganism | CSC Type | Testing Conditions |
|---|---|---|
| P. protegens | NipsIt Inside | On shelf encapsulated |
| strain CHA0 | Flo Rite 1706 | and on seed testing |
| (DSM 19095 | EverGol Energy | Time points: 0 days, |
| | Pro-Ized Red Colorant | 7 days, , 1 month, |
| | Biofilm C1006 | 2 months, 3 months |
| | Peridiam Precise 1006 | |
| | Poncho 600 FS | |

Data from the testing is shown in FIG. 9. FIG. 9 is up to one month.

What is claimed:

1. An encapsulated microorganism, comprising particles comprising a core and a coating;
wherein the core comprises:
a microorganism and/or an enzyme; and
an alginate or polyaspartate; and
wherein the coating comprises a lipid-based coating.

2. The encapsulated microorganism of claim 1 wherein the core further comprises a polyelectrolyte.

3. The encapsulated microorganism of claim 1 wherein the core comprises a Pseudomonas bacteria.

4. The encapsulated microorganism of any of claims 1-3 wherein the core further comprises a lipid.

5. The encapsulated microorganism of any of claims 1-4 wherein the lipid-based coating comprises a naturally occurring wax.

6. The encapsulated microorganism of claim 5 wherein the naturally occurring wax is selected from the group consisting of carnuba wax, sorghum wax, candelilla wax, rice bran wax, soy wax, and combinations thereof.

7. The encapsulated microorganism of claim 5 wherein the lipid-based coating comprises hydrogenated soybean oil blended with stearic acid.

8. The encapsulated microorganism of any of the preceding claims wherein the lipid-based coating contains at least 95 wt % lipids.

7. The encapsulated microorganism of claim 3 wherein the alginate or polyaspartate comprise calcium alginate, sodium alginate, manganese alginate, zirconium alginate, calcium poly(aspartate), manganese poly(aspartate), zirconium poly (aspartate), and combinations thereof. with alginates being particularly preferred.

8. The encapsulated microorganism of claim 3 wherein the alginate or polyaspartate is an alginate cross-linked by MnCl2, BaCl2, CaCl2, or combinations thereof.

9. The encapsulated microorganism of any of the preceding claims wherein the core comprises a microorganism and wherein the encapsulated microorganism and/or enzyme is in the form of particles and wherein at least 90% of the particles are between 1 and 100 μm in diameter.

10. The encapsulated microorganism of any of the preceding claims wherein the encapsulated microorganism is in the form of particles and wherein at least 70% of the particles are between 1 and 10 μm in diameter.

11. The encapsulated microorganism of any of the preceding claims wherein the core comprises nitrogen-fixing bacteria, endo and ectomycorrhizal fungi, and/or plant growth promoting bacteria.

12. The encapsulated microorganism of any of the preceding claims wherein the core comprises: Rhizobium tropici, Rhizobium leguminosarum, Sinorhizobium meliloti, Pseudomonas protegens, Burkholderia phytofirmans, Bradyrhizobium japonicum, Azospirillum brasilense, Rhodopseudomonas acidophila Rhodotorula sp., and/or Penicillium bilaiae.

13. The encapsulated microorganism of any of the preceding claims wherein the encapsulated microorganism and/or enzyme is in the form of particles and wherein, as compared to the uncoated particles, the microoganism's activity is 10 times higher or 100 times higher than the uncoated particles after 8 weeks or after 15 months of storage at standard conditions (20° C. in air, 1 atm, no humidity).

14. The encapsulated microorganism of any of the preceding claims in the form of a coating disposed on a seed.

15. The encapsulated microorganism of any of the preceding claims wherein the core comprises skim milk, trehalose or maltodextrin.

16. The encapsulated microorganism of any of the preceding claims wherein the microorganism exhibits at least 10E6 colony forming units after 1 week or 4 weeks or 12 months, or a 100× or less or 10× or less loss in viability after 1 week or 4 weeks or 12 months when stored at about 20° C. and 50% relative humidity.

17. A seed coated with the encapsulated microorganism of any of the preceding claims.

18. The seed of claim 17 wherein the seed is a seed of corn, wheat, soy bean, or cotton.

19. A method of growing a plant comprising putting one of the seeds of claims 14, 17-18 into soil.

20. A method of making an encapsulated microorganism, comprising:
dropping a solution of the microorganism (or combination of microorganisms) and an alginate and/or polyaspartate into a stirred bath containing a cross-linking solution to form particles comprising the microorganism and a cross-linked alginate or polyaspartate;
coating the resulting particles with a lipid-based wax so that the lipid waxes form an outer shell over the alginate or polyaspartate of the core particle.
